SEQUENCE LISTING (3 sequences)

SEQ ID NO: 1 - DNA, 32 bp, Artificial Sequence
  primer
gccgaagggaatggatactgagggaatagcaa

SEQ ID NO: 2 - DNA, 32 bp, Artificial Sequence
  primer
tcccgagaactctcacaatatccaaacagcag

SEQ ID NO: 3 - DNA, 45 bp, Artificial Sequence
  primer
  misc_feature (28)..(29): There is FAM-dT between nucleotides 28 and 29
  misc_feature (29)..(30): There is THF between nucleotides 29 and 30
  misc_feature (30)..(31): There is BHQ-dT between nucleotides 30 and 31
gaacattacgtcttatgtccagatacgtggccgtgataggagtga

(19) United States
(12) Patent Application Publication
(10) Pub. No.: US 2020/0362399 A1
(43) Pub. Date: Nov. 19, 2020

(54) Method of enhancing isothermal amplification sensitivity of nucleic acid and reagents thereof

(71) Applicant: Hangzhou Zhongce Bio-Sci&Tech. Co., Ltd., Hangzhou (CN)

(72) Inventors: Qi CHENG, Hangzhou (CN); Zhenju HUANG, Hangzhou (CN); Xianbo QIU, HANGZHOU (CN); Jianxun ZHANG, HANGZHOU (CN)

(21) Appl. No.: 16/553,667

(22) Filed: Aug. 28, 2019

(30) Foreign Application Priority Data
May 17, 2019 (CN) 201910414614.7

(51) Int. Cl. C12Q 1/6848; C12Q 1/686; C12Q 1/70

(52) U.S. Cl. CPC C12Q 1/6848; C12Q 1/70; C12Q 1/686

(57) ABSTRACT

Disclosed herein is a method of enhancing RAA isothermal amplification sensitivity using magnetic beads and applications in nucleic acid analysis and detection and medical diagnosis, belonging to the biomedical engineering field, comprising: (1) material selection of magnetic beads, (2) optimization of diameter of magnetic beads, (3) optimization of mixing time of magnetic beads, and (4) sensitivity detection method of magnetic beads for RAA isothermal detection, wherein the material of magnetic bead is steel bead, the diameter of the magnetic bead is 1.5 mm, the mixing time of the magnetic bead is 30 s, and the detection method of isothermal RAA results includes agarose gel and fluorescence detection. Under this condition, the sensitivity of magnetic beads for RAA isothermal amplification is increased by 10 times compared to that without adding magnetic beads, and increased by 100 times compared to that of other types of fluorescence detectors. The method disclosed herein can significantly enhance the sensitivity of RAA isothermal amplification, and have a wide application in rapid detection of nucleic acids and clinical diagnosis.

### CROSS REFERENCE TO RELATED APPLICATION

The present application claims priority of Chinese Patent Application No. 201910414614.7, filed on May 17, 2019. The content of this application including all tables, diagrams and claims is incorporated hereby as reference in its entity.

### FIELD OF THE INVENTION

The present invention relates to recombinase-aid amplification (RAA) reaction in the molecular biology technology field, in particular to a method of enhancing RAA isothermal amplification sensitivity using magnetic beads.

### BACKGROUND OF THE INVENTION

The Recombinase-aid Amplification (RAA) technique is a method of rapid amplification of nucleic acids under a constant temperature. Unlike RPA, the RAA amplification method uses a recombinase obtained from bacteria or fungi. The recombinase can bind tightly to the primer DNA to form a polymer of the enzyme and the primer at a constant temperature of 37° C. When the sequence completely complementary to the template DNA is searched by the primer, the template DNA is melted with the help of a single-stranded DNA binding (SSB), and a new DNA complementary strand is formed under the action of DNA polymerase, and the reaction product also increases exponentially, usually the amplified fragment which can be detected by agarose gel electrophoresis is obtained within 1 h. Fluorophores are added to the RAA reaction system to monitor the entire RAA amplification process in real time using the accumulated fluorescent signals, and the quantitative and qualitative analysis of the starting template can be performed within 20 minutes.

It has been more than 10 years since the development of RAA technique. With the continuous development and advancement of science and technology, isothermal RAA technique has become one of the most important technologies in molecular biology and has been widely used in health and epidemic prevention, genetics, microbiology, etc. However, this technique has not achieved an ideal result in the diagnosis of viruses in clinical medicine and disease incubation period and sometimes even detection is missed. Since virus replicates very little during the disease incubation period and there are no enough nucleic acids to meet the detection limit, virus genes are unable to capture in many conventional detection techniques.

### SUMMARY OF THE INVENTION

There are few studies on methods of enhancing isothermal RAA detection efficiency at home and abroad. Most studies focus on optimizing the efficiency of PCR amplification and enhancing the specificity and yield of PCR by adding formamide, DMSO, glycerol, BSA, nonionic detergent and tetramethylammonium chloride, etc. These methods have no substantial changes to the isothermal RAA amplification system, and even inhibit amplification or cannot achieve amplification.

It was surprisingly found that it is effective to change the sensitivity of the RAA detection by changing the motion and binding efficiency between molecules of the RAA reaction system. It was also surprisingly found that the addition of magnetic bead to the RAA reaction system can significantly change the sensitivity of RAA detection. In view of this, it is an object of the present invention to provide a method of enhancing RAA isothermal amplification sensitivity using magnetic beads and reagents thereof.

To achieve the foregoing object, in one embodiment, the present invention adopts the following technical solutions:

A method of enhancing RAA isothermal amplification sensitivity using magnetic beads and applications in nucleic acid analysis and detection and medical diagnosis, belonging to the biomedical engineering field, comprising: (1) material selection of magnetic beads, (2) optimization of diameter of magnetic beads, (3) optimization of mixing time of magnetic beads, and (4) sensitivity detection method of magnetic beads for RAA isothermal detection, wherein the material of magnetic bead is steel bead, the diameter of the magnetic bead is 1.5 mm, the mixing time of the magnetic bead is 30s, and the detection method of isothermal RAA results includes agarose gel and fluorescence detection. Under this condition, the sensitivity of magnetic beads for RAA isothermal amplification is increased by 10 times compared to that without adding magnetic beads, and increased by 100 times compared to that of other types of fluorescence detectors.

Further, for the method of enhancing isothermal RAA amplification sensitivity, wherein the material of magnetic beads is chosen from one or more of Teflon, polyethylene, polypropylene, iron beads, steel beads, tungsten steel beads and nickel beads. In some preferred embodiments, the most preferred material is steel bead.

In some embodiments, for the method of enhancing isothermal RAA amplification sensitivity, wherein the magnetic bead is subjected to rinsing, sterilization, ultrasonication, drying, etc.

In some embodiments, for the method of enhancing isothermal RAA amplification sensitivity, wherein the diameter of the magnetic bead can be 1 mm, 1.5 mm, 2 mm, 2.5 mm, etc., most preferably 1.5 mm.

In some embodiments, for the method of enhancing isothermal RAA amplification sensitivity, wherein the mixing time of magnetic beads in the reaction tube is 5 s, 10 s, 15 s, 20 s, 25 s, 30 s, etc., most preferably 20s.

In some embodiments, for the method of enhancing isothermal RAA amplification sensitivity, wherein the RAA amplification comprises: conventional RAA, low copy RAA amplification, long fragment RAA amplification, complex template RAA amplification, asymmetric primer RAA amplification, fluorescent RAA amplification, reverse transcription RAA amplification and repeated amplification RAA.

In some embodiments, the kit and the method of enhancing isothermal RAA amplification sensitivity are used in the biomedical fields such as public health and epidemic prevention, clinical diagnosis, and disease-related genetic analysis, etc.

In some embodiments, for the use, the clinical disease diagnosis is infectious disease diagnosis, sexually transmitted disease diagnosis and cancer diagnosis, etc.

In one aspect, the invention provides a method of isothermal amplification of nucleic acid, comprising: adding magnetic beads in an amplification reagent, wherein the diameter of the beads is from 0.5 mm to 3 mm.

Preferably, the magnetic beads are selected from the group consisting of Teflon, polyethylene, polypropylene, iron beads, steel beads, tungsten steel beads and nickel beads.

Preferably, the beads are magnetic steel beads.

Preferably, the beads have a diameter of 1 to 1.5 mm.

Preferably, the beads have a diameter of 1.5 mm.

Preferably, before the nucleic acid amplification, the magnetic bead and the amplification reagent are in a solution state, and the nucleic acid reagent solution is mixed for 5 to 40 seconds. Preferably, the mixing time is 20 to 30 seconds.

Preferably, when the magnetic bead is a steel bead, the mixing time is 20 seconds.

Preferably, the magnetic bead is subjected to rinsing, sterilization, ultrasonication, drying, etc. before contact with a nucleic acid amplification reagent.

Preferably, the nucleic acid amplification method comprises a RAA and/or a RPA method.

Preferably, the nucleic acid is a nucleic acid of African swine fever.

Preferably, the volume ratio of the magnetic bead to the amplification reagent solution is 1:1 to 1:3.

In another aspect, the present invention provides a RAA amplification reagent, wherein the reagent comprises a reagent necessary for nucleic acid amplification, wherein the reagent further comprises a magnetic bead.

Preferably, the magnetic beads are selected from the group consisting of Teflon, polyethylene, polypropylene, iron beads, steel beads, tungsten steel beads and nickel beads.

Preferably, the beads are magnetic steel beads.

Preferably, the beads have a diameter of 1 to 1.5 mm.

Preferably, the beads have a diameter of 1.5 mm.

Preferably, when the amplification reagent is a solution, the volume ratio of the magnetic bead to the amplification reagent solution is 1:1 to 1:3.

### BRIEF DESCRIPTION OF THE DRAWINGS

FIG. 1 is an experiment diagram of screening magnetic beads.

FIG. 2 is a comparison experiment diagram of magnetic bead diameter.

FIG. 3 and FIG. 4 are experiment diagrams of pre-mixing time of magnetic beads.

FIG. 5 and FIG. 6 show the comparisons of sensitivity with magnetic beads and without magnetic beads, wherein FIG. 5 represents an experimental result with magnetic beads, FIG. 6 represents an experimental result without magnetic beads.

### DETAILED DESCRIPTION

### Detection and Assay

Detection means assaying or testing the presence or absence of a substance or a material, such as, but not limited to, chemicals, organic compounds, inorganic compounds, metabolites, drugs or drug metabolites, organic tissues or metabolites thereof, nucleic acids, proteins or polymers. In addition, detection means testing the amount of a substance or a material.

### Recombinase Polymerase Amplification

The Recombinase-aid Amplification (RAA) technique is a method of rapid amplification of nucleic acids under a constant temperature. Unlike RPA, the RAA amplification method uses a recombinase obtained from bacteria or fungi. The recombinase can bind tightly to the primer DNA to form a polymer of the enzyme and the primer at a constant temperature of 37° C. When the sequence completely complementary to the template DNA is searched by the primer, the template DNA is melted with the help of a single-stranded DNA binding (SSB), and a new DNA complementary strand is formed under the action of DNA polymerase, and the reaction product also increases exponentially, usually the amplified fragment which can be detected by agarose gel electrophoresis is obtained within 1 h. Fluorophores are added to the RAA reaction system to monitor the entire RAA amplification process in real time using the accumulated fluorescent signals, and the quantitative and qualitative analysis of the starting template can be performed within 20 minutes.

In the present invention, RAA and RPA are interchangeable, and the addition of magnetic bead is understood to have similar effect on enhancing the sensitivity of amplification.

RPA or RAA is a method of nucleotide amplification (for example, isothermal amplification). Generally, in the first step of RPA, a recombinase contacts a first and a second nucleotide primers to form a first and a second nucleoprotein primers. In the second step, the first and second nucleoprotein primers are in contact with a double-stranded nucleotide template to form a first double-stranded structure in the first portion of the first strand of the template nucleotide, and to form a second double-stranded structure in the second portion of the second strand of the template nucleotide. For example, in a given DNA molecule, the 3′ ends of the first nucleotide primer and the second nucleotide primer are opposite each other. Generally, in the third step, the 3′ ends of the first and the second nucleoprotein primers are amplified by a DNA polymerase to produce a first and a second double-stranded nucleotides, and a first and a second unsubstituted nucleotide strands. Generally, the second step and the third step can be repeated until the amplification reaches the expected level.

As described herein, the enzyme used by RPA or RAA, known as a recombinase, is capable of pairing oligonucleotide primers homologous to a double-stranded DNA template. In this manner, DNA is synthesized in a double-stranded DNA template. In the presence of a nucleotide template, an exponential amplification reaction is initiated using two or more sequence-specific primers (for example, gene-specific). The reaction is rapid and the result of specific amplification of the double-stranded DNA template sequence is that the DNA template is amplified from only a few copies to a detectable level within a few minutes. The RPA method has been disclosed, for example, disclosed in the U.S. Pat. Nos. 7,270,981, 7,399,590, 7,666,598, 7,435,561, the U.S. Patent Publication No. US 2009/0029421, and international application WO 2010/141940. All of these documents are used as a part of the present invention and are incorporated by reference.

The RPA or RAA reaction involves the coordination of various proteins and other factors, just as supporting DNA synthesis from pairing the 3′ end of the oligonucleotide to the complementary substrate, these other factors support the activity of recombinant elements in the system. In some embodiments, the RPA reaction comprises a mixture of a recombinase, a single-stranded binding protein, a polymerase, dNTPs, ATP, a primer, and a nucleotide template. In some embodiments, the RPA reaction may include one or more of the following substances (in any combination): at least one recombinase; at least one single-stranded binding protein; at least one DNA polymerase; dNTPs or a mixture of dNTPs and ddNTPs; a crowding agent; a buffer; a reducing agent; an ATP or an analog of ATP; at least one recombinant loaded protein; a first primer and any second primer; a probe; a reverse transcriptase; and a nucleotide template molecule, such as a single-stranded (for example, RNA) or a double-stranded nucleotide. In some embodiments, the RPA reaction may include, for example, a reverse transcriptase. Examples of other RPA reaction mixtures are not limited to those described herein.

In some embodiments, the RPA or RAA reaction may comprise a UvsX protein, a gp32 protein and a UvsY protein. The components, microparticles or methods described herein may include, or partially include, for example, UvsX protein, gp32 protein and UvsY protein. For example, the components, microparticles, or any of the methods described herein may comprise, or partially comprise, for example, T6H66S UvsX, Rb69 gp32 and Rb69 UvsY.

In some embodiments, the RPA or RAA reaction can comprise a UvsX protein and a gp32 protein. For example, any component, any microparticle or any method described herein may comprise, or partially comprise, for example, UvsX protein and gp32 protein.

One protein component of the RPA or RAA reaction is a recombinase, which may be from a prokaryote, a virus or a eukaryote. Typical recombinases comprise RecA and UvsX (for example, the RecA protein or UvsX protein obtained from any species), and their fragments or mutants, and any combination thereof. RecA and UvsX proteins can be obtained from any species. RecA and UvsX fragments or mutant proteins can also be produced by appropriate RecA and UvsX proteins, nucleotide sequences and molecular biology techniques (for example, refer to the formation of UvsX mutants as described in U.S. Pat. No. 8,071,308). Typical UvsX proteins include those derived from the myoviridae phages, for example, T4, T2, T6, Rb69, Aeh1, KVP40, Acinetobacter phage 133, Aeromonas phage. 65, cyanophage P-SSM2, cyanophage PSSM4, cyanophage S-PM2, Rb14, Rb32, Aeromonas phage 25, Vibrio phage nt-1, phi-1, Rb16, Rb43, phage 31, phage 44RR2.8t, Rb49, phage Rb3, and phage LZ2. Other typical recombinase proteins include archaeal RADA and RADB proteins and eukaryotic (e.g., plant, mammalian, and fungal) Rad51 proteins (e.g., RAD51, RAD51B, RAD51C, RAD51D, DMC1, XRCC2, XRCC3, and recA) (see Lin et al., Proc. Natl. Acad. Sci. U.S.A. 103:10328-10333, 2006).

In any of the methods of the present invention, the recombinase (e.g., UvsX) may be a mutant or hybrid recombinase. In some embodiments, the UvsX mutant is Rb69 UvsX, which comprises at least one mutation in its amino acid sequence, wherein the mutant may be selected from the group consisting of amino acid mutations, and the amino acid mutations may include: (a) the amino acid at the site 64 is serine but not histidine, with one or more glutamic acid residues added to the C-terminus, one or more aspartic acids added to the C-terminus, or any combination therebetween. In other embodiments, the UvsX mutant is T6 UvsX having at least one T6 UvsX amino acid sequence mutation, wherein the mutant is selected from an amino acid mutation group. The amino acid mutations may include: (a) the amino acid at the site 66 is not histidine, (b) the amino acid at the site 66 is serine, (c) one or more glutamic acid residues are added to the C-terminus, (d) one or more aspartic acid is added to the C-terminus, and (e) any combination therebetween. Where a hybrid recombinant protein is used, the hybrid protein may, for example, be UvsX protein whose amino acid sequences of at least one region are from different species. The region may be, for example, a DNA-binding loop-2 binding domain of UvsX.

In addition, one or more single-stranded DNA binding proteins can be used to stabilize the nucleotides during various continuous exchange reactions. The one or more single-stranded DNA binding proteins may be derived from or obtained from a variety of species, such as from prokaryotes, viruses or eukaryotes. Typical single-stranded DNA binding proteins include, but are not limited to, E. coli SSB and those single-stranded DNA binding proteins derived from myovirus phage, such as T4, T2, T6, Rb69, Aeh1, KVP40, Acinetobacter phage 133, Aeromonas phage 65, cyanophage P-SSM2, cyanophage PSSM4, cyanophage S-PM2, Rb14, Rb32, Aeromonas phage 25, Vibrio phage nt-1, phi-1, Rb16, Rb43, phage 31, phage 44RR2.8t, Rb49, phage Rb3, and phage LZ2. Examples of additional single-stranded DNA-binding proteins include A. denitrificans Alide-2047, Burkholderia thailandensis BthaB 33951, Prevotella pallens HMPREF9144-0124, and replication protein A eukaryotic single-stranded DNA binding proteins.

The DNA polymerase can be a polymerase of eukaryotic or prokaryotic organisms. Examples of eukaryotic polymerases include pol-alpha, pol-beta, pol-delta, pol-epsilon, and mutants or fragments thereof, or combinations thereof. Examples of prokaryotic polymerases include E. coli DNA polymerase I (for example, Klenow fragment), phage T4 gp43 DNA polymerase, large fragment of Bacillus stearothermophilus polymerase I, Phi-29 DNA polymerase, T7 DNA polymerase, Bacillus subtilis Pol I, Staphylococcus aureus Pol I, E. coli DNA polymerase I, E. coli DNA polymerase II, E. coli DNA polymerase III, E. coli DNA polymerase IV, E. coli DNA polymerase V, and mutants or fragments thereof, or combinations thereof. In some embodiments, the DNA polymerase lacks 3′-5′ exonuclease activity. In some embodiments, the DNA polymerase has a strand displacement function, for example, large fragments of type I and PolV prokaryotic polymerases.

Any of the methods of the present invention can be carried out in the presence of a crowding agent. In some embodiments, the crowding agent may include one or more of polyethylene glycol/polyoxyethylene, polyethylene oxide, polyvinyl alcohol, polypropylene, polysucrose, dextran, polyethylene (vinyl pyrrolidone) (PVP) and albumin. In some embodiments, the molecular weight of the crowding agent is not more than 200,000 Daltons. The crowding agent is present in a weight/volume ratio (w/v) of about 0.5% to about 15%.

Recombinant loading proteins, when used, may be derived from a prokaryote, a virus or a eukaryote. Typical recombinant loading proteins include E. coli RecO, E. coli RecR, UvsY, and mutants or fragments thereof, or combinations thereof. Typical UvsY proteins include those derived from myoviridae phases such as T4, T2, T6, Rb69, Aeh1, KVP40, Acinetobacter phage133, Aeromonas phage 65, cyanophage P-SSM2, cyanophage PSSM4, cyanophage S-PM2, Rb14, Rb32, Aeromonas phage 25, Vibrio phage nt-1, phi-1, Rb16, Rb43, phage 31, phage 44RR2.8t, Rb49, phage Rb3 and phage LZ2. In any of the methods of the invention, the recombinant loading reagent can be from myoviridae phage. Myoviridae phage may be, for example, T4, T2, T6, Rb69, Aeh1, KVP40, Acinetobacter phage133, Aeromonas phage 65, cyanophage P-SSM2, cyanophage PSSM4, cyanophage S-PM2, Rb14, Rb32, Aeromonas phage 25, Vibrio phage nt-1, phi-1, Rb16, Rb43, phage 31, phage 44RR2.8t, Rb49, phage Rb3 and phage LZ2.

Additionally, any method of the present invention can be performed with interruption primers. The interruption primer is a primer that does not allow the polymerase to extend. When interruption primer is used, de-interruption reagent can be used to initiate the primer and allow extension. The de-interruption reagent can be an endonuclease or exonuclease for primer-breaking. Typical de-interruption reagents include E. coli exonuclease III and E. coli endonuclease IV.

In some embodiments, the present invention comprises: the recombinase is contacted with the first and second nucleotide primers and the third extension interruption primer to form a first and a second and a third nucleic acid protein primers, wherein the interruption primers contain one or more non-complementary or modified residues; the first and second nucleic acid protein primers are in contact with the double-stranded target nucleotide, forming a first double-stranded structure between the first nucleic acid protein primer of the first strand (forming the D-loop) and the first strand of the DNA, and forming a second double-stranded structure between the second nucleic acid protein primer of the second strand (forming the D-loop) and the second strand of the DNA, by this way, the first nucleic acid protein primer and the second nucleic acid protein primer are oriented toward each other on the same target nucleotide of third part of target nucleotide located between the 5′ end of the first primer and the 5′ end of the second primer. One or more polymerases and dNTPs are used to extend the 3′ ends of the first nucleic acid primer and the second nucleic acid primer, thereby forming a first amplification of the target nucleotide; in the presence of the nuclease, the first amplification of the target nucleotide contacts the third nucleic acid primer at the target nucleoside to form a third double-stranded structure (forming a D-loop), only when the third double-stranded structure is formed, the nuclease specifically disassembles the non-complementary internal residues to form a third 5′ primer and a third 3′ extension interruption primer; and one or more polymerases and dNTPs are used to extend the 3′ ends of the third 5′ primer to produce a second double-stranded amplified nucleotide.

In some embodiments, the methods comprise: the first and the second primers amplify a first portion of the double-stranded target nucleotide to produce a first amplification product, and at least one additional primer can be used to amplify part of continuous sequence within the first amplification product (for example, an additional third primer is used to bind the first or second primer to amplify part of continuous sequence within the first amplification product). In some embodiments, the method comprises: the first and the second primers amplify a first portion of the double-stranded target nucleotide to produce a first amplification product, and the third and fourth primers can be used for amplifying part of continuous sequence within the first amplification product.

In some embodiments, the method includes, for example, a forward primer and a reverse primer. In some embodiments, the amplification method comprises at least one interruption primer comprising one or more non-complementary or internally modified residues (e.g. one or more non-complementary or internal modified residues that can be recognized and cleared by nucleases, and the nucleases can be, for example, DNA glycosylase, depurinated pyrimidine (AP) endonuclease, fpg, Nth, MutY, MutS, MutM, E. coli MUG, human MUG, human Ogg1, vertebrate Nei-like (Neil) glycosylase, Nfo, exonuclease III, or urinary glycosylase). Other nucleotide examples (for example, primers and probes) are not limited to the methods described herein.

In some embodiments, the amplification method may include a primer or probe that is resistant to a nuclease, for example, comprising at least one (e.g., at least 2, 3, 4, 5, 6, 7, or 8) phosphorothioate linkages.

Any of the processes of the present invention can be performed in the presence of heparin. Heparin, as a reagent for reducing non-specific primer impurities, can increase the function of E. coli exonuclease III or E. coli endonuclease IV to rapidly eliminate the 3′ interrupter or the terminal residue from a recombinant intermediate.

Depending on the particular type of reaction, the mixture may comprise one or more buffers, one or more salts, and one or more nucleotides. The reaction mixture can be continued within a particular temperature or temperature range that favors the reaction. In some embodiments, the temperature is maintained at or below 80° C., for example, at or below 70° C., at or below 60° C., at or below 50° C., at or below 40° C., at or below 37° C., at or below 30° C., and at or below room temperature. In some embodiments, the temperature is maintained at or above 4° C., at or above 4° C. 10° C., at or above 15° C., at or above 20° C., at or above 25° C., at or above 30° C., at or above 37° C., at or above 40° C., at or above 50° C., at or above 60° C., at or above 70° C. In some embodiments, the reaction mixture is maintained at room temperature or ambient temperature. In some embodiments, the change in Celsius temperature of the mixture over the entire reaction time is less than 25% (e.g., less than 20%, less than 15%, less than 10%, less than 5%) and/or the change in Celsius temperature of the mixture over the entire reaction time is less than 15° C. (e.g., less than 10° C., less than 5° C., less than 2° C., or less than 1° C.).

Detection of amplification, for example, real-time detection, can be performed by methods well known in the art. In some embodiments, one or more primers or probes (for example, molecular beacon probes) are labeled with one or more detectable labels. Typical detectable labels include enzymes, enzyme substrates, coenzymes, enzyme inhibitors, fluorescent labels, quenchers, luminophores, magnetic powder or glass beads, redox-sensitive groups (electrochemically active groups), luminescent labels, radioisotopes (including radionucleotides), and binding pair member. More specific examples include fluorescence, algal protein, tetraethyl rhodamine and β-galactosamine. Binding pair members may include biotin/avidin, biotin/streptavidin, antigen/antibody, ligand/receptor, and derivatives and mutants of these binding pairs. It should be noted here that the fluorescent quencher is considered a detectable label. For example, a fluorescent quencher can be contacted with a fluorescent dye and the amount of quenching can be detected.

### Magnetic Beads and Use

The beads referred to herein refers to beads with a certain diameter and a certain volume. In some embodiments, these beads are metal beads, for example, iron, copper, steel, nickel beads, etc. In some other embodiments, the beads may also be non-metallic beads. In some embodiments, beads may be magnetic beads. The magnetic beads generally refer to the beads whose motion may be influenced in magnetic fields, for example, beads that can be magnetized. In some embodiments, beads themselves are magnetic, and beads can be influenced in a magnetic field. In some embodiments, the influence is the ability to move the beads under the action of a magnetic field. In some embodiments, the magnetic beads may be mixed with a nucleic acid reaction reagent, and the nucleic acid reaction reagent is an isothermal amplification reagent. In some embodiments, the isothermal amplification reagents include reagents necessary for PRA or RAA amplification.

In some embodiments, the magnetic bead may be pre-mixed with the isothermal amplification reagent, and then amplified, or the magnetic bead may be mixed with the reagent to allow the magnetic bead to remain in the amplification solution during amplification. The reagents herein are generally solution reagents. Of course, when preparing an amplification reagent, the magnetic bead may be pre-mixed with the reagent, and then prepared as a dry powder reagent. When actual amplification is necessary, the reagent to be amplified is prepared into a liquid for amplification.

In some embodiments, the magnetic bead herein has a diameter of about 1.5 mm, which may be 0.1 mm, 0.2 mm, 0.5 mm, 0.6 mm, 0.7 mm, 0.8 mm, 0.9 mm, 1 mm, 1.2 mm, 1.3 mm, 1.4 mm, 1.5 mm, 1.6 mm, 1.7 mm, 1.8 mm.

In some embodiments, when the amplification reagent is a solution, there are one or more magnetic beads, and the ratio of volume of the magnetic bead to the solution may be 1:2.5, 1:1, 1:2.8, 1:3.0, 1:3.2, 1:0.8, 1:4.0, etc.

In some embodiments, the magnetic bead is pre-treated to remove impurities or oil stains on the surface of the bead, and these substances may interfere with the amplification of nucleic acids. In some embodiments, the magnetic bead is a steel bead having a diameter of 1.5 mm.

In some embodiments, before or during nucleic acid amplification, the magnetic bead is pre-mixed with the nucleic acid amplification reagent and then amplified. The pre-mixing time may be 5 seconds, 10 seconds, 15 seconds, 20 seconds, 25 seconds, 30 seconds, 35 seconds. In some embodiments, the magnetic bead may be moved in a solution while mixing, and the purpose of the movement is to mix the solution more evenly. The nucleic acid amplification reagent may contain interfering substance that affects the amplification reaction. The treatment methods may include washing, decontaminating, drying, disinfecting, sterilizing, etc.

In some embodiments, the movement of magnetic beads or the pretreatment of the magnetic beads with the amplification reagents are performed in an isothermal amplification detector that has the function of heating and maintaining a constant temperature and reading fluorescence. Of course, it can be appreciated that the functions of magnetic field generation, temperature control, and fluorescence reading may be independent or combined, for example, having the functions of magnetic field generation, temperature control, and fluorescence reading simultaneously. For example, a real-time fluorescence isothermal amplification detector can achieve rapid nucleic acid amplification for isothermal amplification reagents; at the same time, real-time fluorescence signal acquisition and processing can be carried out during the amplification reaction, and negative/positive detection results are interpreted. At the same time, high-precision temperature control heating is performed on a plurality of reaction tubes, and the temperature uniformity between respective hole positions is ensured by an optimized design. The fluorescent dye inside the reaction tube can also be fluorescently excited by a high-intensity LED of a specific wavelength, and at the same time, a high-sensitivity photodiode (PD) is used to receive the emitted fluorescent signal; in particular, the optimized spatial light path design may achieve the most optimal fluorescence detection performance, ensuring the detection sensitivity of the instruments. In addition, the magnetic beads inside the reaction tube can be manipulated by the periodically changing strong magnetic field provided by the moving magnet, and the periodic up and down movement of magnetic beads in the reaction reagent may be used to enhance the internal mixing of the reaction reagents and improve the reaction efficiency.

The mechanism that the magnetic beads improve the sensitivity of detection is still not known at present, but it may be related to the viscous reaction reagents and tiny bubbles occurring after the reaction. By adding magnetic beads or particles, these phenomena can be effectively improved, which ultimately increases the sensitivity of isothermal amplification.

### Specimens and Samples

Specimens that can be detected by a detection device of the present invention include biological fluids (e.g., case fluids or clinical specimens). Liquid or fluid samples can be derived from solid or semi-solid samples, including excreta, biological tissue and food samples. Solid or semi-solid samples can be converted to liquid samples by any suitable method, such as mixing, mashing, maceration, incubation, dissolving or digesting solid samples by enzymolysis in a suitable solution (e.g., water, phosphate solution or other buffer solution). “Biological samples” include samples derived from animals, plants and foods, including, for example, urine or saliva, blood and its components from human or animals, spinal fluid, vaginal secretions, sperm, feces, sweat, secretions, tissues, organs, tumors, cultures of tissues and organs, cell cultures and media. The preferred biological sample is urine. Food samples include food processed materials, final products, meat, cheese, wine, milk and drinking water. Plant samples include those derived from any plant, plant tissue, plant cell culture and medium. “Environmental samples” are derived from the environment (e.g., liquid samples from lakes or other bodies of water, sewage samples, soil samples, groundwater, seawater, and waste liquid samples). Environmental samples may also include sewage or other wastewater.

### Analytes

The analytes referred to herein are nucleic acid substances in a sample. The nucleic acid herein may be a nucleic acid substance in any living substance such as a virus, a bacterium, a tissue, etc. The nucleic acid herein may be DNA, RNA, etc.

Examples of microorganisms whose genome consists of DNA and can be used for e amplification analysis of the present invention include, but not limited to, Aspergillus, Aspergillus flavus, Aspergillus fumigatus, Aspergillus nidulans, Candida, Cryptococcus neoformans, Cryptococcus gilchristi, cryptococcosis, herpes virus, hepatitis B virus, herpes simplex virus 1-2, human cytomegalovirus, Mycoplasma pneumoniae, human herpesvirus, JC virus, Papillomavirus 1-82, parvovirus B, pseudopox virus, SV40 virus, vaccinia virus, varicella-zoster virus, and variola virus. Some microorganisms may have undergone genomic evolution, which has led to resistance to some therapeutic treatments that are effective for their wild-type counterparts. In this case, multiple assays are often required to detect infectious microorganisms. The subtypes of those microorganisms and specific resistant strains provide diagnostic information for therapeutic targeting. A method capable of simultaneously detecting infectious microorganisms, subtypes, and resistant strains will improve the time and cost required for diagnosis.

Viruses whose genomes are composed of RNAs and whose RNAs are required to converted to cDNA by reverse transcriptase prior to PCR include, but are not limited to, astrovirus, Bunia virus, California encephalitis virus, St. Louis encephalitis virus, West Nile virus, Japanese encephalitis virus, Eastern equine encephalitis virus, western equine encephalitis virus, Venezuelan equine encephalitis virus, Murray Valley encephalitis virus, Chikungunya virus, tick fever virus, hemorrhagic fever virus, Coxsackie virus A 1-24, Coxsackie virus B1-6, dengue virus 1-4, Duvenhage virus, eastern equine encephalitis virus, Ebola virus, echo virus 1-24, enterovirus 1-71, intestinal Coronavirus, Hantavirus, Hepatitis A virus, Hepatitis C virus, E virus, Human immunodeficiency virus (HIV) 1 and 2, Respiratory coronavirus, Rotavirus, T-lymphocyte virus, Influenza A, Influenza Virus B, Junin virus, Lassa fever virus, measles virus, mumps virus, Norwalk virus, lymphocytic choriomeningitis virus, parainfluenza virus 1-4, poliovirus 1-3, Rabies virus, respiratory syncytial virus, rhinovirus 1-113, Rocio virus, rubella virus, vesicular stomatitis virus, yellow fever virus, Zika virus.

Examples of the present invention can be used to detect or screen a variety of diseases or pathological conditions, such as cancer. Cancers that can be assessed by the methods and components of the invention include cancer cells, including cells and cancer cells from the bladder, blood, bone, bone marrow, brain, breast, colon, esophagus, gastrointestinal, gums, head, kidney, lung, nasopharynx, neck, ovaries, pancreas, prostate, skin, stomach, testes, tongue or uterus.

Furthermore, it has been confirmed that cancers have the following histological types, although they are not limited to: magligant tumors, carcinoma; undifferentiated carcinoma; giant cell and spindle cell carcinoma; small cell carcinoma; papillary carcinoma; squamous cell carcinoma; lymphatic epithelial carcinoma; basal cell carcinoma; transitional cell carcinoma; papillary transitional cell carcinoma; adenocarcinoma; gastrin cholangiocarcinoma; hepatocellular carcinoma; combined hepatocellular carcinoma and cholangiocarcinoma; trabecular gland cancer; adenoid cystic carcinoma; adenomatous polyp adenocarcinoma; adenocarcinoma, familial colon polyps; solid cancer; carcinoid; malignant tumor. Branch alveolar adenocarcinoma; papillary adenocarcinoma; pigmented carcinoma; eosinophilic carcinoma; eosinophilic adenocarcinoma; basophilic squamous cell carcinoma; clear cell adenocarcinoma; granulosa cell carcinoma; follicular adenocarcinoma; papillary and follicular adenocarcinoma; non-envelopic sclerosing carcinoma; adrenal cortical carcinoma endometrial cancer; cutaneous adenocarcinoma; apical adenocarcinoma; sebaceous gland cancer; cervical adenocarcinoma; mucoepidermoid carcinoma; cystic adenocarcinoma; papillary cystadenocarcinoma; Serous cystic adenocarcinoma; mucinous cystadenocarcinoma; mucinous adenocarcinoma; signet ring cell carcinoma; invasive ductal carcinoma; medullary carcinoma; lobular carcinoma; inflammatory cancer; Paget's disease, breast; acinar cell carcinoma; Adenosquamous carcinoma;

adenocarcinoma/squamous metaplasia; thymoma ovarian stromal tumor, malignant; Malignant neoplasms; granulosa cell tumor, malignant; glioblastoma; sertoli cell carcinoma; lymphocyte tumor lipid cell tumor, malignant; paraganglioma extramammary glioma, malignant; Chromoblastoma; mesangial malignant melanoma; leukocytic melanoma; superficial diffuse melanoma; giant melanoma epithelioid cell melanoma; blue sarcoma; fibrosarcoma; fibroblastoma mucinous sarcoma; liposarcoma; leiomyosarcoma rhabdomyosarcoma; embryonic rhabdomyosarcoma; alveolar rhabdomyosarcoma; interstitial sarcoma mixed tumor; mullerian mixed tumor; nephroblastoma; hepatoblastoma; carcinosarcoma; stromal tumor; Brenner tumor; lobular tumor; synovial sarcoma; mesothelioma; clonal embryonal carcinoma; teratoma, ovarian cancer choriocarcinoma; middle renal angiosarcoma; hemangioendothelioma, malignant; Kaposi's sarcoma; vascular epithelioma, lymphatic sarcoma osteosarcoma; proximal osteosarcoma; chondrosarcoma chondroblastoma interstitial chondrosarcoma; giant cell tumor of bone; Ewing's sarcoma; odontogenic tumor, malignant; ameloblastic sarcoma; tumor ameloblastic fibrosarcoma; Pineal chordoma glioma ependymoma astrocytoma primary astrocytoma; fibrogenic astrocytoma; astrocytoma; glioblastoma; oligodendroglioma; oligodendroglioma; primitive neuroectodermal cerebellar sarcoma neuroblastoma; Neuroblastoma; retinoblastoma; olfactory neurogenic tumor; meningioma neurofibrosarcoma; schwannomas granulosa cell tumor, malignant; malignant lymphoma Hodgkin's disease; Hodgkin's lymphoma; accessory nerve malignant lymph Neoplasms, small lymphocytes; malignant lymphoma, large cells, diffuse; malignant lymphoma, follicles; mycosis fungoides; other designated non-Hodgkin's lymphoma; malignant histiocytosis; multiple myeloma; mast cells sarcoma; immunoproliferative small bowel disease; leukemia; lymphoid leukemia; plasma cell leukemia; erythroleukemia lymphosarcoma leukemia; myelogenous leukemia; alkaloid leukemia; eosinophilic leukemia; monocytic leukemia; mast cell leukemia; megakaryocyte leukemia; myeloma; and hairy cell leukemia. In addition, genetic mutations and alterations at the RNA level are assessed as precancerous latency, such as transformation, abnormal structure, and hyperplasia.

### Beneficial Effects

1. The present invention uses magnetic beads to increase the sensitivity of RAA isothermal amplification sensitivity by 10 to 100 times compared with the conventional RAA detection techniques; 2. The present invention relates to the method of enhancing RAA isothermal amplification sensitivity by magnetic beads. The magnetic beads used herein are cheap and easily available, easy to store at a room temperature for a long time and convenient to use. 3. The RAA isothermal amplification sensitivity of the present invention is significantly improved, so that the applications for RAA isothermal amplification are more efficient and purposeful. It is of great theoretical significance and application value for public health and epidemic prevention, clinical diagnosis and disease-related gene analysis, etc.

The present invention is further illustrated by the following specific embodiments but is not limited thereto. The scope of protection of the present invention is defined by scope claimed in the claims.

### Example 1: Preparation of Reagents and Samples and Setting of Reaction Conditions

1. Templates, Primers and Probes

Preparation of template: A plasmid cloned from the African swine fever VP72 gene (commercially available) was used. The concentration of extracted template DNA was determined by Nanodrop and converted to 2.54×1010 copies/μl, then diluted to 1000 copies/μl, 100 copies/μl, 10 copies/μl, 1 copy/μl with sterile deionized water, as the reaction templates of the experiment. The e African swine fever VP72 gene was amplified by the following primers:

VP72-RAA-F:
(SEQ ID No: 1)
GCCGAAGGGAATGGATACTGAGGGAATAGCAA
VP72-RAA-R:
(SEQ ID No: 2)
TCCCGAGAACTCTCACAATATCCAAACAGCAG
VP72-RAA-P:
(SEQ ID No: 3)
GAACATTACGTCTTATGTCCAGATACGT[FAM-dT]G[THF]G
[BHQ-dT]CCGTGATAGGAGTGA.

Sterile deionized water was used as a negative control. The above primers, positive samples and negative controls were all verified under PCR fluorescence reaction conditions, and they could be detected normally, indicating that the primers could be used. The specific verification process was omitted here.

2. Reagents

PEG and MgAC were purchased from Sigma and prepared by selves.

TABLE 1
The reagent formulation

| RAA reaction system components | Volume (μL) |
|---|---|
| RAA reagent dry powder | Prepare in advance and freeze the |
| | powder in a reaction tube |
| A Buffer | 12.5 μL |
| B Buffer | 2.5 μL |
| Primer mixture | 4 μL |
| Specific fluorescent probe | 0.6 μL |
| DNA template | 2 μL |
| ddH2O | 28.4 μL |
| Total volume | 50 μL |

A Buffer was 20% PEG, prepared with sterile ultrapure water, pH was not adjusted deliberately;

B Buffer was 280 mM MgAc, prepared with sterile ultrapure water, with a natural pH;

The components of the RAA dry powder reagent were as follows: 1 mmol/L dNTP, 90 ng/μL SSB protein, 120 ng/μL recA recombinase protein (SC-recA/BS-recA), 30 ng/μL Rad51, 30 ng/μL Bsu DNA polymerase, 100 mmol/L Tricine, 20% PEG, 5 mmol/L dithiothreitol, 100 ng/μL creatine kinase, Exo exonuclease.

3. Preparation of RAA reaction system: Each test sample included a negative control corresponding to one RAA reaction dry powder tube. The reaction components and volumes added in each RAA reaction dry powder tube were shown in Table 1.

4. Reaction Conditions

Constant temperature fluorescence detector: Genchek-2 (Hangzhou ZC Bio-Sci&Tech Co. Ltd.)

Reaction conditions: 37° C.,

Reaction time: 20 min;

### Example 2: Pre-Treatment of Magnetic Beads

The steel beads, iron beads, nickel beads or plastic beads that were just purchased had oil or other impurities, which should be pre-treated. The specific process was as follows:

1) The beads were placed in a plastic bottle, and the volume ratio of the round beads to the plastic bottle was 1:3;

2) Clear water was added, and the bottle lid was covered, then the plastic bottle was shaken for 5 min, and then washed repeatedly for 3 times;

3) Deionized water was added to repeat the step 2 for 1-2 times until the wastewater after washing was free of floating matter.

4) Deionized water was added and placed to an ultrasonic instrument for cleaning for 1 h, and the ultrasonic power was set to high-grade, with temperature set between 50° C. and 60° C.;

5) The round beads were taken out and placed into a plastic bottle, autoclaved at 121° C. for 25 min;

6) The sterilized steel beads were placed in an oven for drying and standby;

### Example 3: Screening of Different Magnetic Beads

The African swine fever viruses of 100 copies/μl were used as positive samples and the deionized water was used as a negative control to investigate the effect of magnetic beads with different properties on the detection sensitivity.

The specific RAA reagents and reaction conditions were the same. Reagents were prepared according to table 1 as described in Example 1. Only the positive samples had different magnetic beads, and the magnetic beads were divided into: magnetic iron beads, magnetic steel beads, magnetic nickel beads, and plastic magnetic beads, which were purchased from Mingliang Steel Beads Factory, Mingliang Steel Beads Factory, Nangong Casting Alloy Material Co., Ltd. and Thermo Fisher, respectively, with a particle diameter of 1.5 mm.

These magnetic beads were firstly treated according to Example 2, and then one bead was added to the 8-row tube of the RAA dry powder, and then reagents in the Table 1 were prepared into reaction solution for reaction. Before reaction, the magnetic beads were moved back and forth in the solution or shaken for 20 seconds and then a formal amplification reaction was performed.

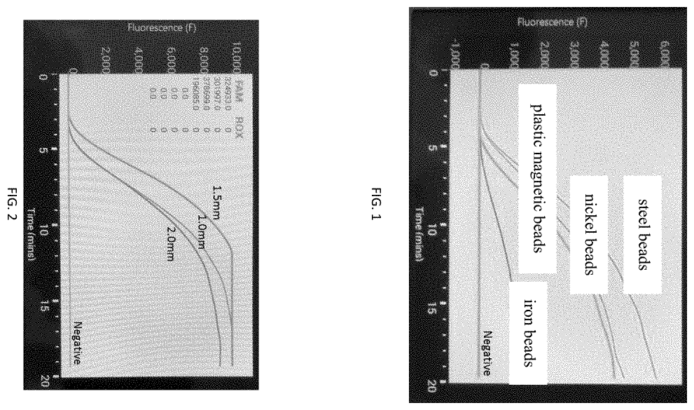

As can be seen from the results of FIG. 1, the amplification curve of magnetic steel beads was the most typical, with obvious exponential and plateau periods, high fluorescence intensity (ordinate value), and small CT value (abscissa corresponding to the intersection of curve and threshold line). Compared with tungsten steel beads, magnetic steel beads had no difference in peak time, but there was a certain difference in fluorescence value, and the fluorescence intensity was relatively weak. Other magnetic beads, such as iron beads and plastic magnetic beads had lower rise, with larger CT values, and non-obvious plateau period. In our initial experiments, some beads did not show amplification and were missed detection, such as cobalt beads, etc.

It indicated that the magnetic steel beads made the RAA isothermal amplification product to have faster replication speed, more quantity, and higher amplification reaction efficiency, with better utilization value.

The CT values of different magnetic bead as follows:

| Magnetic bead | CT value | Fluorescence value |
|---|---|---|
| Magnetic iron bead | 7.45 | 1200 |
| Magnetic steel bead | 3.61 | 5900 |
| Magnetic nickel bead | 3.62 | 4500 |
| Plastic magnetic bead | 5.68 | 4200 |

### Example 4: Screening of Diameter of Magnetic Beads

The African swine fever viruses of 100 copies/μl were used as positive samples and the deionized water was used as a negative control. The specific reagents and reaction conditions were the same as those described in Examples 1 and 2, except that the diameters of the magnetic beads were 1 mm, 1.5 mm, and 2 mm.

As shown from the result of FIG. 2, when the template concentration was the same and the diameter of the magnetic bead was 1.5 mm, the amplification curve of RAA was most typical, with obvious exponential and plateau periods, and higher fluorescence intensity (ordinate value), and the CT value was small, which was 3.12 (the abscissa corresponding to the intersection of the curve and the threshold line). When the magnetic bead was' mm and 2 mm, there was no difference in peak time, but the peak time was later than that of the 1.5 mm steel beads, so magnetic beads of 1.5 mm in diameter were selected for subsequent studies. It indicated that the size of the diameter has an effect.

### Example 5: Optimization of Pre-Mixing Time of Magnetic Beads

The mixing time of the magnetic beads in the present invention can be 10 s, 20 s, 30 s, and the optimal mixing time was 20 s (mixing time referred to the time for mixing after sample was prepared and magnetic bead was added. Once the mixing time was satisfied, real RAA amplification reaction was started). The magnetic bead here had a diameter of 1.5 mm. The other conditions were the same as those in Example 4, except that the duration of mixed contact was different.

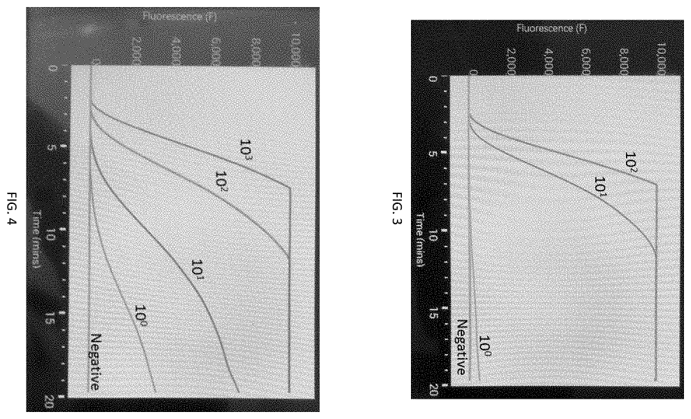

As shown from FIG. 3 and FIG. 4, the template concentrations were 100 copies/μl, 10 copies/μl, 1 copy/μl. In case of 100 copies/μl and 10 copies/μl, the mixing time of 20 s and 30 s had no significant difference in the effect on RAA amplification stability. However, in case of 1 copy/μl, the peak time of RAA amplification for mixing time of 30 s was significantly later than that of mixing time of 20 s, and the fluorescence value was low, affecting the detection sensitivity. For samples of 1 copy/μl, after mixing 30 s, reaction was started and 1 copy/μl of sample could not be effectively detected, but samples of 100 copies/μl, 10 copies/μl could be detected (FIG. 3), in fact, the test result of 1 copy/μl was equivalent to the negative result, resulting in the missed detection of the samples of low concentration. Therefore, it was best to select the premixing time for 20 s, and it could detect 1 samples of 1 copy/μl, enhancing the sensitivity (see FIG. 4). It could effectively distinguish the negative sample and the low concentration samples of 1 copy/μl, significantly improving the sensitivity of the detection.

### Example 6: Comparison of RAA Detection Sensitivity with Magnetic Beads and without Magnetic Beads

Magnetic beads having a diameter of 1.5 mm were used, and one bead was added to each RAA dry powder reaction tube. The specific RAA reagents and reaction conditions were the same as above. The components were prepared according to the Table 1.

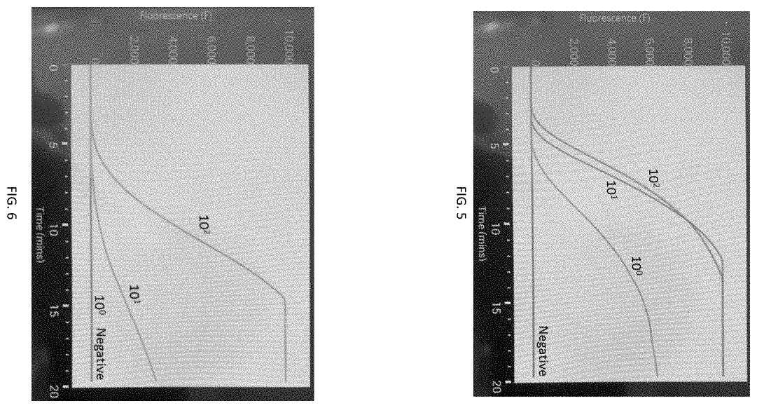

As shown from FIG. 5 and FIG. 6, when the template concentration was 100 copies/μl, 10 copies/μl, 1 copy/μl, the sensitivity of RAA isothermal fluorescence amplification could reach a single copy in the case of adding steel beads (1 copy/μl), having normal “S” curve and high fluorescence value.

In contrast, the RAA isothermal amplification sensitivity without addition of magnetic beads was 10 copies/μl and the peak time was later than that of the amplification with magnetic beads, and the single-copy samples could not be detected. Therefore, the addition of magnetic beads could increase the sensitivity of RAA isothermal amplification by 10 times.

The invention shown and described herein may be implemented in the absence of any elements, limitations specifically disclosed herein. The terms and expressions used herein are for illustration rather than limitation, which do not exclude any equivalents of the features and portions described herein in the use of these terms and expressions, in addition, it should be understood that various modifications are feasible within the scope of the present invention. It is therefore to be understood that, although the invention has been particularly disclosed by various embodiments and alternative features, modifications and variations of the concepts described herein may be employed by those of skilled in the art, and such modifications and variations will fall into the scope of protection of the present invention as defined by the appended claims.

The contents of the articles, patents, patent applications, and all other documents and electronic information available or documented herein are incorporated herein by reference in their entirety, as if each individual publication is specifically and individually indicated for reference. The applicant reserves the right to incorporate any and all materials and information from any such article, patent, patent application or other document into this application.

1. A method of isothermal amplification of nucleic acids in a sample, comprising: a reagent necessary for amplification of nucleic acids, wherein a magnetic bead is added to the amplification reagent, and the magnetic bead has a diameter of 0.5 mm to 3 mm.

2. The method according to claim 1, wherein the magnetic bead is selected from the group consisting of Teflon, polyethylene, polypropylene, iron beads, steel beads, tungsten steel beads and nickel beads.

3. The method according to claim 1, wherein the magnetic bead is a magnetic steel bead.

4. The method according to claim 1, wherein the magnetic bead has a diameter of 1 to 1.5 cm.

5. The method according to claim 3, wherein the magnetic bead has a diameter of 1.5 cm.

6. The method according to claim 1, wherein before the nucleic acid amplification, the magnetic bead and the amplification reagent are in a solution state, and the nucleic acid reagent solution is mixed for 5 to 40 seconds.

7. The method according to claim 1, wherein the mixing time is 20 to 30 seconds.

8. The method according to claim 1, wherein when the magnetic bead is a steel bead, the mixing time is 20 seconds

9. The method according to claim 8, wherein the nucleic acid is a swine fever virus nucleic acid.

10. The method according to claim 1, wherein the magnetic bead is subjected to rinsing, sterilization, ultrasonication, and drying before contact with a nucleic acid amplification reagent.

11. The method according to claim 1, wherein the nucleic acid amplification method comprises a RAA and/or a RPA method.

12. The method according to claim 1, wherein the nucleic acid is a nucleic acid of African swine fever.

13. The method according to claim 6, wherein the volume ratio of the magnetic bead to the amplification reagent solution is 1:1 to 1:3.

14. A reagent for RAA amplification of nucleic acids in samples, wherein the reagent comprises a reagent necessary for nucleic acid amplification, wherein the reagent further comprises a magnetic bead.

15. The reagent according to claim 14, wherein the magnetic bead is selected from the group consisting of Teflon, polyethylene, polypropylene, iron beads, steel beads, tungsten steel beads and nickel beads.

16. The reagent according to claim 14, wherein the bead is a magnetic steel bead.

17. The reagent according to claim 14, wherein the magnetic bead has a diameter of 1 to 1.5 mm.

18. The reagent according to claim 17, wherein the magnetic bead has a diameter of 1.5 mm.

19. The reagent according to claim 14, wherein when the amplification reagent is a solution, the volume ratio of the magnetic bead to the amplification reagent solution is 1:1 to 1:3.

20. The reagent according to claim 14, wherein the reagent necessary for nucleic acid amplification comprises recombinase, single-stranded binding protein and polymerase.
